SEQUENCE LISTING (10 sequences)

SEQ ID NO: 1 - DNA, 1857 bp, Bacillus thuringiensis
  CDS (1)...(1857)
atggagataagtgaccagaatcaatacatcccctataactgtttgaataatcctgaaagtgagatatttaatgctagaaattccaatttcggactggtttctcaagtcagctcgggacttacgcgttttcttctagaggcagctgtcccagaggctggttttgcacttggcctattcgatatcatttggggcgctctaggcgttgatcaatggagcctatttcttaggcacattgagcaattaatacgacaagaaataacagagttagaaaggaatagagcgactgcaatattaactggactatcgtcaagctataatctatatgttgaggcgttaagagaatggcaaaatgatcctaataatccagcctcacaagaaagagtacgtacacgttttcgtctaacggacgacgctatagtaacaggtttacctactttggcaattcggaatcttgaggtagtgaatttatcagtctatactcaagcagcaaatctacacttatctttgttaagagatgccgtttactttggagaaagatggggattaacacaagcaaatattgaagatctgtacacaagactcacgagtaatatccaagaatattcagaccattgtgcaagatggtataatcaaggtttaaatgagattggagggataagtaggagatatttggacttccaaagagattttacaatttctgtcttagatattgtcgcccttttcccaaattacgatatccgaacatatcctattccgacacaaagtcaatttacaagggagatttatacctctcccgtcgttgcaggtaatataaattttggtttaagtatagcgaatgtattgagagcccctcatctgatggactttattgatcggatagtcatttatacaaattcagttagaagtactccatattgggcagggcatgaagtcatattgagaagaacagggcaagggcaaggaaatgagataagatttcctttatatggagtggctgcaaatgcagaaccaccagttactataagacctacaggatttactgatgagcaacgacaatggtatagagcgcgatcgcgtgttgttttgtttagaagttcaggtcaagactttagtttggtagatgccgtaggattccttactatatttagcgctgtttcaatctatagaaatggctttggatttaacactgatactattgatgaaattccaattgagggaaccgatccattcactggatatagccaccgattatgccatgtgggctttcttgcgtcatctccattcatcagtcagtatgcaagggctcctatattttcttggacgcaccgtagtgcaacccttacaaatacaatggctccagatgtcattacccaaataccgttagtaaaggctttcaatcttcattcaggtgccacgattgttaaaggaccaggttttacaggtggggatatccttcgaagaacgaatgttggtagctttggagatatgcgtgtaaacattactgcaccactatcacaaagatatcgcgtaaggattcgttatgcttctacgacagatttacaattctatacgaatattaatggaactactattaatattggtaatttctcgagcactatggacagtggggatgatttacagtacggaagattcagggttgcaggttttactactccatttaccttttcagatgcaaacagcacattcacaataggtgcttttggcttctctccaaacaacgaagtttatatagatcgaattgaatttgtcccggcagaagtaacatttgaggcagaatatgatttagagaaagctcagaaagcggtgaatgcgctgtttacttcttccaatcaatag

SEQ ID NO: 2 - protein, 618 aa, Bacillus thuringiensis
  DOMAIN (1)...(618): Deduced amino acid sequence Cry1M
MEISDQNQYIPYNCLNNPESEIFNARNSNFGLVSQVSSGLTRFLLEAAVPEAGFALGLFDIIWGALGVDQWSLFLRHIEQLIRQEITELERNRATAILTGLSSSYNLYVEALREWQNDPNNPASQERVRTRFRLTDDAIVTGLPTLAIRNLEVVNLSVYTQAANLHLSLLRDAVYFGERWGLTQANIEDLYTRLTSNIQEYSDHCARWYNQGLNEIGGISRRYLDFQRDFTISVLDIVALFPNYDIRTYPIPTQSQFTREIYTSPVVAGNINFGLSIANVLRAPHLMDFIDRIVIYTNSVRSTPYWAGHEVILRRTGQGQGNEIRFPLYGVAANAEPPVTIRPTGFTDEQRQWYRARSRVVLFRSSGQDFSLVDAVGFLTIFSAVSIYRNGFGFNTDTIDEIPIEGTDPFTGYSHRLCHVGFLASSPFISQYARAPIFSWTHRSATLTNTMAPDVITQIPLVKAFNLHSGATIVKGPGFTGGDILRRTNVGSFGDMRVNITAPLSQRYRVRIRYASTTDLQFYTNINGTTINIGNFSSTMDSGDDLQYGRFRVAGFTTPFTFSDANSTFTIGAFGFSPNNEVYIDRIEFVPAEVTFEAEYDLEKAQKAVNALFTSSNQ

SEQ ID NO: 3 - DNA, 17 bp, Artificial Sequence
  M13 Forward Primer
gtaaaacgacggccagt

SEQ ID NO: 4 - DNA, 16 bp, Artificial Sequence
  M13 Reverse Primer
aacagctatgaccatg

SEQ ID NO: 5 - DNA, 30 bp, Artificial Sequence
  Cry1M-376-F Primer
gaaagagtacgtacacgttttcgtctaacg

SEQ ID NO: 6 - DNA, 30 bp, Artificial Sequence
  Cry1M-1269-R Primer
gcatactgactgatgaatggagatgacgcc

SEQ ID NO: 7 - protein, 618 aa, Bacillus thuringiensis
  DOMAIN (1)...(618): Crystal protein
MEISDQNQYIPYNCLNNPESEIFNARNSNFGLVSQVSSGLTRFLLEAAVPEAGFALGLFDIIWGALGVDQWSLFLRQIEQLIRQEITELERNRATAILTGLSSSYNLYVEALREWENDPNNPASQERVRTRFRLTDDAIVTGLPTLAIRNLEVVNLSVYTQAANLHLSLLRDAVYFGERWGLTQANIEDLYTRLTSNIQEYSDHCARWYNQGLNEIGGISRRYLDFQRDLTISVLDIVALFPNYDIRTYPIPTQSQLTREIYTSPVVAGNINFGLSIANVLRAPHLMDFIDRIVIYTNSVRSTPYWAGHEVISRRTGQGQGNEIRFPLYGVAANAEPPVTIRPTGFTDEQRQWYRARSRVVSFRSSGQDFSLVDAVGFLTIFSAVSIYRNGFGFNTDTIDEIPIEGTDPFTGYSHRLCHVGFLASSPFISQYARAPIFSWTHRSATLTNTIAPDVITQIPLVKAFNLHSGATIVKGPGFTGGDILRRTNVGSFGDMRVNITAPLSQRYRVRIRYASTTDLQFYTNINGTTINIGNFSSTMDSGDDLQYGRFRVAGFTTPFTFSDANSTFTIGAFGFSPNNEVYIDRIEFVPAEVTFEAEYDLEKAQKAVNALFTSSNQ

SEQ ID NO: 8 - protein, 613 aa, Artificial Sequence
  Synthetic construct
MEISDQNQYIPYNCLNNPESEIFNARNSNFGLVSQVSSGLTRFLLEAAVPEAGFALGLFDIIWGALGVDQWSLFLRQIEQLIRQEITELERNRATAILTGLSSSYNLYVEALREWENDPNNPASQERVRTRFRLTDDAIVTGLPTLAIRNLEVVNLSVYTQAANLHLSLLRDAVYFGERWGLTQANIEDLYTRLTSNIQEYSDHCARWYNQGLNEIGGISRRYLDFQRDTISVLDIVALFPNYDIRTYPIPTQSQTREIYTSPVVAGNINFGLSIANVLRAPHLMDFIDRIVIYTNSVRSTPYWAGHEVIRRTGQGQGNEIRFPLYGVAANAEPPVTIRPTGFTDEQRQWYRARSRVVFRSSGQDFSLVDAVGFLTIFSAVSIYRNGFGFNTDTIDEIPIEGTDPFTGYSHRLCHVGFLASSPFISQYARAPIFSWTHRSATLTNTAPDVITQIPLVKAFNLHSGATIVKGPGFTGGDILRRTNVGSFGDMRVNITAPLSQRYRVRIRYASTTDLQFYTNINGTTINIGNFSSTMDSGDDLQYGRFRVAGFTTPFTFSDANSTFTIGAFGFSPNNEVYIDRIEFVPAEVTFEAEYDLEKAQKAVNALFTSSNQ

SEQ ID NO: 9 - protein, 620 aa, Bacillus thuringiensis
  DOMAIN (1)...(620): Delta-endotoxin
MEISDQNQYIPYNCLNNPESEIFNARNSNFGLVSQVSSGLTRFLLEAAVPEAGFALGLFDIIWGALGVDQWSLFLRQIEQLIRQEITELERNRATAILIGLSSSYNLYVEALREWENDPNNPASQERVRTRFRLTDDAIVTGLPTLAIRNLEVVNLSVYTQAANLHLSLLRDAVYFGERWGLTQANIEDLYTRLTSNIQEYSDHCARWYNQGLNEIGGISRRYLDFQRDLTISVLDIVAPFSPNYDIRTYPIPTQSQLTREIYTSPVVAGNINFGLSIANVLRAPHLMDFIDRIVIYTNSVRSTPYWAGHEVISRRTGQAQGNEIRFPLYGVAANAEPPVTIRPTGFTDEQRQWYRARSLLSRFRSSGQDFSLVDAVAFLTIFSAVSIYRNGFGFNTDTIDEIPIEGTDPFTGYSHRLCHVGFLASSPFISQYARAPIFSWTHRSATLTNTIAPDVITQIPLVKAFNLHSGATIVKGPGFTGGDILRRTNVRSFRDMRVNITAPLSQRYRVRIRYASTTDLQFYTNINGTTINIGNFSSTMDSGDDLQYGRFRVAGFTTPFTFSRCKQTFHNRSFWFSPKLTEVYIDRIEFVPAEVTFEAEYDLEKAQKAVNALFTSSNQ

SEQ ID NO: 10 - protein, 596 aa, Artificial Sequence
  Synthetic construct
MEISDQNQYIPYNCLNNPESEIFNARNSNFGLVSQVSSGLTRFLLEAAVPEAGFALGLFDIIWGALGVDQWSLFLRQIEQLIRQEITELERNRATAILGLSSSYNLYVEALREWENDPNNPASQERVRTRFRLTDDAIVTGLPTLAIRNLEVVNLSVYTQAANLHLSLLRDAVYFGERWGLTQANIEDLYTRLTSNIQEYSDHCARWYNQGLNEIGGISRRYLDFQRDLTISVLDIVAPFPNYDIRTYPIPTQSQTREIYTSPVVAGNINFGLSIANVLRAPHLMDFIDRIVIYTNSVRSTPYWAGHEVIRRTGQQGNEIRFPLYGVAANAEPPVTIRPTGFTDEQRQWYRARSLFRSSGQDFSLVDAVFLTIFSAVSIYRNGFGFNTDTIDEIPIEGTDPFTGYSHRLCHVGFLASSPFISQYARAPIFSWTHRSATLTNTAPDVITQIPLVKAFNLHSGATIVKGPGFTGGDILRRTNVSFDMRVNITAPLSQRYRVRIRYASTTDLQFYTNINGTTINIGNFSSTMDSGDDLQYGRFRVAGFTTPFTFSTFFFSPEVYIDRIEFVPAEVTFEAEYDLEKAQKAVNALFTSSNQ

(19) United States
(12) Patent Application Publication
(10) Pub. No.: US 2009/0098051 A1
(43) Pub. Date: Apr. 16, 2009

(54) BACILLUS THURINGIENSIS TOXIN

(76) Inventors: Mohamed A. IBRAHIM, Frisco, TX (US); Lee A. BULLA, JR., Tioga, TX (US)

Correspondence Address: MORRISON & FOERSTER LLP, 12531 HIGH BLUFF DRIVE, SUITE 100, SAN DIEGO, CA, 92130-2040, US

(21) Appl. No.: 12/172,209

(22) Filed: Jul. 11, 2008

Related U.S. Application Data
(60) Provisional application No. 60/949,156, filed on Jul. 11, 2007.

(51) Int. Cl. A61K 49/00; C07K 14/00; C12N 15/11; C12N 5/04; A01H 5/00; C12P 21/04; C07K 16/18; A61K 38/16

(52) U.S. Cl. 424/9.1; 530/350; 536/23.7; 435/419; 800/302; 435/69.1; 530/387.9; 514/12

(57) ABSTRACT

A new Bt Cry toxin, Cry1M is able to exert insecticidal effects on insects and/or nematodes in general, and in addition, on insects that have acquired resistance to alternative Cry proteins.

### CROSS-REFERENCE TO RELATED APPLICATION

This application claims priority from U.S. provisional application Ser. No. 60/949,156 filed 11 Jul. 2007, the contents of which are incorporated herein by reference.

### TECHNICAL FIELD

The invention relates to a newly discovered gene encoding a Cry1M toxin that has not been previously reported. This gene is useful to confer insect resistance on plants, especially against those insects that have, themselves, become resistant to previously described Bacillus thuringiensis toxins.

### BACKGROUND ART

There are a large number of toxins derived from Bacillus thuringiensis (Bt) that are able to confer insect resistance on plants. The proteins encoded by these “Cry” genes, and themselves entitled “Cry” toxins have been used to provide plants with a defense mechanism against insect damage. Some of the genes encoding these Cry toxins have also been modified to provide them with codons that are preferred for expression in plants.

The various Cry toxins have been named systematically beginning with Cry1A and proceeding through Cry1B, Cry1C, etc. The present invention provides a new toxin, designated herein “Cry1M” that is able to confer resistance on plants, even to insects that have become inured to the activity of previously known Cry toxins.

The nucleotide sequence encoding the protein was checked against sequences available in GenBank, one sequence 95% identical, and another sequence 99% identical were found. However, no insecticidal activity of any protein encoded by these sequences has been shown.

The B. thuringiensis strain from which the nucleotide sequence encoding the invention protein was isolated is described in U.S. Pat. Nos. 5,986,177; 6,210,953; and 6,232,439. This strain, known as B. thuringiensis C-18 was deposited 31 Dec. 1996 with the American Type Culture Collection, which, at the time of deposit, had an address at 10801 University Boulevard, Manassas, Va. 20110, and has ATCC Accession No. 55922. These patents report the isolation of a gene encoding a 719-amino acid protein which was designated Cry1I and is reported toxic against insects from the orders lepidoptera, diptera and coleoptera as well as nematodes. Root worms as targets were named specifically.

The present invention concerns an additional toxin that is produced by Bt strain C-18.

### DISCLOSURE OF THE INVENTION

The invention provides a new Bt toxin, Cry1M, which has the amino acid sequence shown in FIG. 1. Thus, in one aspect, the invention is directed to the Cry1M protein of FIG. 1 and to variants at least 90% identical that are toxic to insects, in purified or isolated form.

In another aspect, the invention is directed to a nucleic acid molecule comprising a nucleotide sequence encoding the Cry1M protein of FIG. 1 or said variants and to recombinant materials for the production of this protein, especially in plants. In other aspects, the invention is directed to plants modified to contain the recombinant materials for the production of the Cry1M protein as described above.

Thus, the invention is also directed to a method to confer insect resistance on plants by modifying them to contain an expression system for a protein that has the 618 amino acid sequence set forth in FIG. 1 or a protein that is at least 90% sequence identity to said protein and retains the ability to be toxic to insects. The invention also relates to plants that have been modified by this method.

In still another aspect, the invention includes methods to ascertain a profile of toxicity for the proteins of the invention by assessing a panel of insects against said proteins. Insecticidal compositions containing this protein are also included within the scope of the invention.

The recombinant materials for production of the Cry1M toxin of the invention are not limited to those operable in plants as it may be desirable to produce the protein for use in insecticidal compositions or in the assay methods of the invention. Thus, the recombinant materials include those that are generally operable in procaryotic or eucaryotic host cells, including unicellular organisms.

In addition, antibodies may be generated to the proteins of the invention and are useful as aids in the purification thereof. Standard immunological techniques may be employed and the antibodies include polyclonal, monoclonal, chimeric, single chain, Fv antibodies and the like. Thus “antibodies” includes not only intact antibodies and immunologically active fragments thereof, but modified forms that are recombinantly produced. The antibodies may be produced in a variety of ways once they are generated and manipulated and thus, the scope of the invention also includes cells that are used to generate such antibodies, such as immortalized B cells, hybridomas, transformed recombinant hosts and the like.

### BRIEF DESCRIPTION OF THE DRAWINGS

FIG. 1 shows the nucleotide sequence of the gene contained in Bacillus thuringiensis that encodes the Cry1M protein of the invention and the deduced amino acid sequence.

FIG. 2 is a series of BLAST searches based on Query 1 (i.e., the 618 amino acid Cry1M protein of the invention).

FIG. 3 shows a gel on which the amplified full length Cry1M gene is detected.

FIGS. 4a and 4b show the purification and detection of recombinant Cry1M protein. FIG. 4a is an SDS PAGE analysis showing the presence of Cry1M toxin in the insoluble and soluble fractions from E. coli modified to produce this protein. FIG. 4b shows a western blot wherein this protein is detected in both soluble and insoluble fractions with anti-Cry1Ab antibody.

FIGS. 5a and 5b show the results of a toxicity test of recombinant Cry1M to kill tobacco hornworm. FIG. 5a is the test assay and FIG. 5b shows the control.

### MODES OF CARRYING OUT THE INVENTION

The invention provides a Cry1M protein and variants thereof previously not associated with insecticidal and/or nematicidal activity. Plants modified to produce this protein will be resistant to insects and for nematodes, even those that have acquired resistance to previously known Cry Bt toxins. Thus, the invention provides a number of materials and applications.

First, the protein itself, either isolated directly from Bacillus thuringiensis or prepared recombinantly or otherwise synthetically, is useful to assess which insects or nematodes will be susceptible to its toxicity. Thus, in a simple assay procedure, the purified protein can simply be used to identify the insects and/or nematodes to which it is toxic as using known assay techniques for insecticidal or nematicidal activity.

The protein itself may also be used in an insecticidal or nematicidal composition for application to agricultural environments or for household or other commercial use. Such compositions may include insect attractants and other excipients that are customary in such compositions.

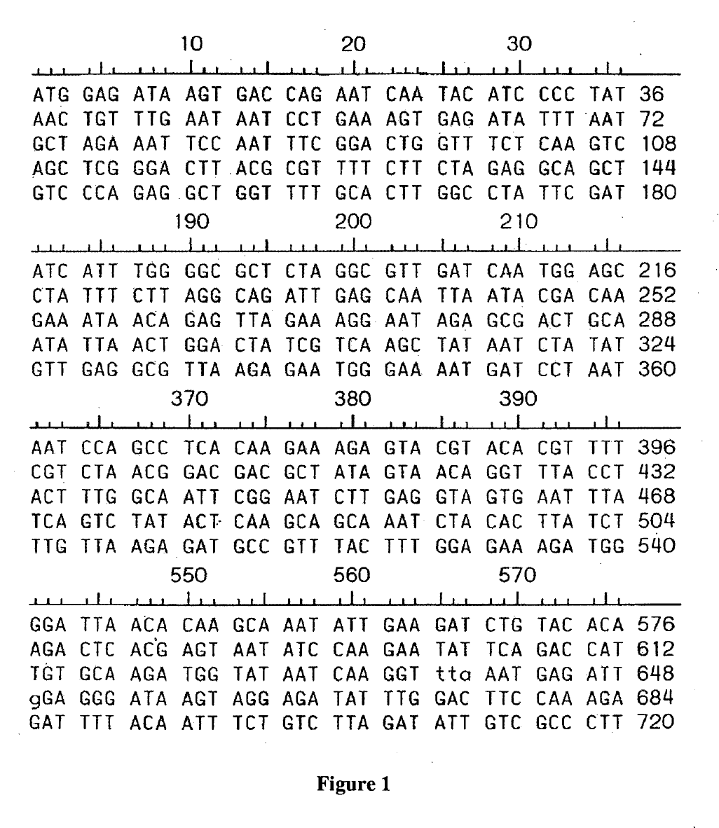

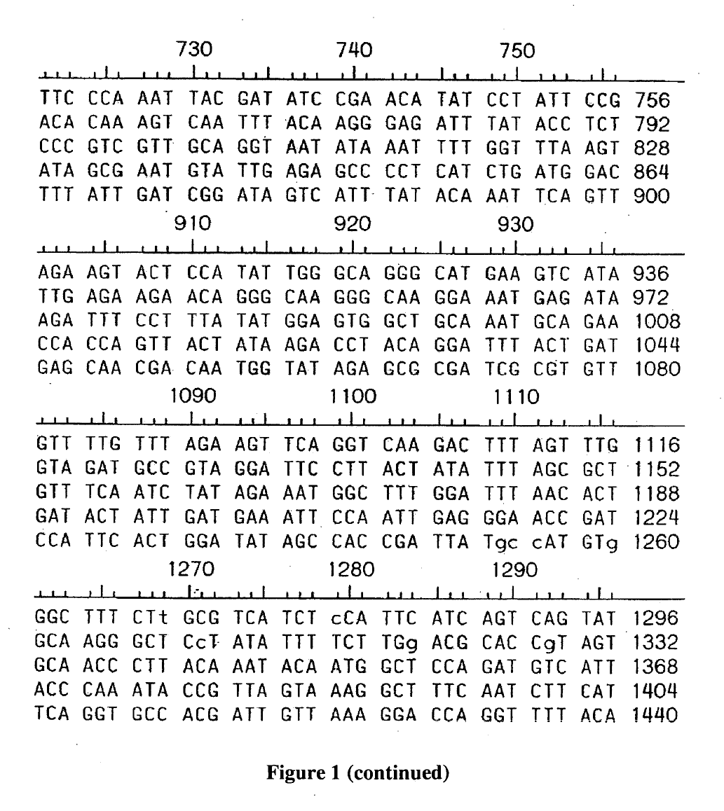

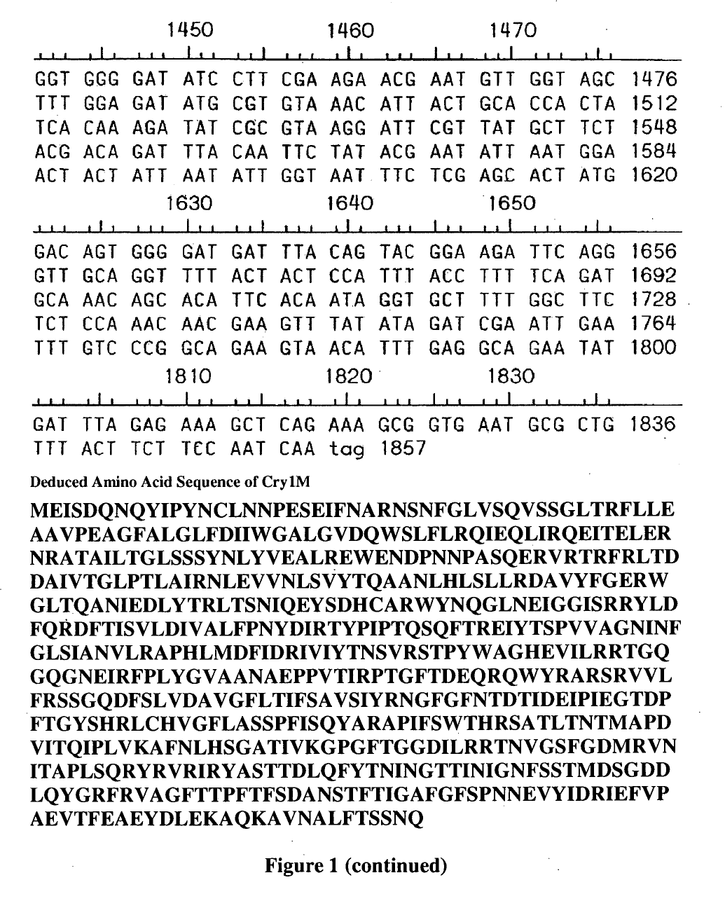

Thus, the invention also relates to insecticidal compositions and/or that are toxic to nematodes containing the protein of the invention. In these compositions, the protein is added in purified and isolated form and is supplemented with excipients customary in such compositions. The amino acid sequence of the protein may be that of the Cry1M protein shown in FIG. 1 or may be a protein with amino acid sequence at least 90%, preferably 93%, more preferably 95%, more preferably 98% or 99% identical to that of the Cry1M protein of FIG. 1, which variant retains insecticidal and/or nematicidal activity.

In another aspect, the invention provides transgenic plants that have acquired insect resistance by virtue of their ability to produce the Cry1M toxin of the invention. In order to prepare such plants, an expression system for the Cry1M toxin is provided to the plant. The modification of the plant may be done by standard techniques, including Agrobacterium transformation, lipofection, electroporation or transfection of plant cells with or without cell walls that are then regenerated into intact plants. A wide variety of methods to modify the genome composition of plants is well known in the art. The expression systems will contain plant-compatible promoters, which may be inducible, tissue specific, constitutive, tissue non-specific or otherwise operable as desired.

The nucleotide sequence encoding the Cry1M toxin may be modified from that shown in FIG. 1 to contain codons whose expression is favorable in plants. Methods for synthesizing sequences of the required length are well known, so any arbitrary sequence that encodes the Cry1M proteins of the invention may be used. This nucleotide sequence is then coupled to appropriate control sequences as noted above for expression in plant cells and intact plants. The Cry1M protein encoded will have the amino acid sequence of the 618 amino acid sequence in FIG. 1 or is a variant thereof that is at least 90%, 93%, 95%, 98% or 99% identical thereto, and which retains insecticidal activity.

The plants modified to contain expression systems for the protein of the invention are resistant to insects in general, and in particular, to insects that may have acquired resistance to other Bt Cry proteins. The range of insects susceptible to the use of the protein per se as an insecticide is expanded by virtue of the lack of previous use of these proteins for insecticidal purposes. Thus, the invention provides insecticides and transformation plants that are resistant to a spectrum of insects not previously susceptible to other Cry proteins or other insecticidal compositions.

As noted above, the protein itself may be used as an insecticide/nematocide and it is conveniently produced recombinantly using the materials described in the present application. Means for recombinant expression in general are by now routine in the art, and expression systems may be designed for operation in prokaryotes such as E. coli and B. subtilis, for eukaryotic unicellular organisms such as yeast and other fungi, and for cell cultures derived from higher organisms such as insect cells, mammalian cells, or avian cells. A plethora of suitable promoters, enhancers, terminating sequences, and other elements of nucleic acids useful to control expressions in various organisms.

When the protein is prepared so as to permit preparation of formulations for application of the protein to plants or to other environments where insect control or nematode control is desired, the protein itself is preferably purified using general techniques well known in the art, using various chromatographic and other purification techniques. A useful reagent for affinity purification includes the use of antibodies directed against the Cry1M toxin or fragments thereof with immunological activity.

Such antibodies can be prepared by conventional means by immunizing animals and harvesting polyclonal antibodies from serum or by preparing monoclonal cultures from these systems. B cells producing the antibodies may be immortalized and used to produce such monoclonal antibodies or the nucleotide sequences encoding them may be isolated and the antibodies or their fragments subsequently produced recombinantly.

Thus, the antibodies of the invention include all forms that are immunoreactive with the Cry1M toxin including Fab, F(ab′)₂ fragments, monoclonal antibodies characteristic of the immunized animal, chimeric antibodies containing the constant region from one animal and the variable region from another, various single-chain forms produced recombinantly and the like.

The antibodies may also be used to assay the levels of the Cry1M protein in a sample and to monitor the levels produced by plants that have been genetically altered to produce this protein. Thus, plants that are modified to produce the protein may be extracted and the extracts tested using the antibodies of the invention as defined above. A wide variety of immunological techniques is available for such testing, such as radioimmunoassays, ELISA assays, and the like including homogeneous embodiments thereof.

In summary, the invention proteins may be used themselves as insecticides and nematicides and the recombinant materials for their production may be used to modify plants to confer insect resistance.

The Cry1M protein has already been verified to be effective against a variety of insects and nematodes, including corn earworm (Helicoverpa zea); black cut worm (Agrotis ipsilon); cabbage looper (Trichoplusia ni); saltmarch caterpillar (Estigmene acrea); tobacco budworm (Heliothis virescens); and lettuce armyworm (Pseudaletia unipuncta).

Other suitable targets may be identified using the assay methods of the invention.

The following examples are intended to illustrate but not to limit the invention.

### EXAMPLE 1

### Recovery of the Cry1M-Encoding DNA

Total genomic DNA from B. thuringiensis subsp. g. alleria (C18) was packaged in M13 phage vector pENTR™/SD/D-TOPO (InvitroGen) The cry1M gene was amplified using standard PCR procedures employing the following primers:

M13 Forward
GTAAAACGACGGCCAGT
M13 Reverse
AACAGCTATGACCATG
cry1M-376-F
GAAAGAGTACGTACACGTTTTCGTCTAACG
cry1M-1269-R
GCATACTGACTGATGAATGGAGATGACGCC

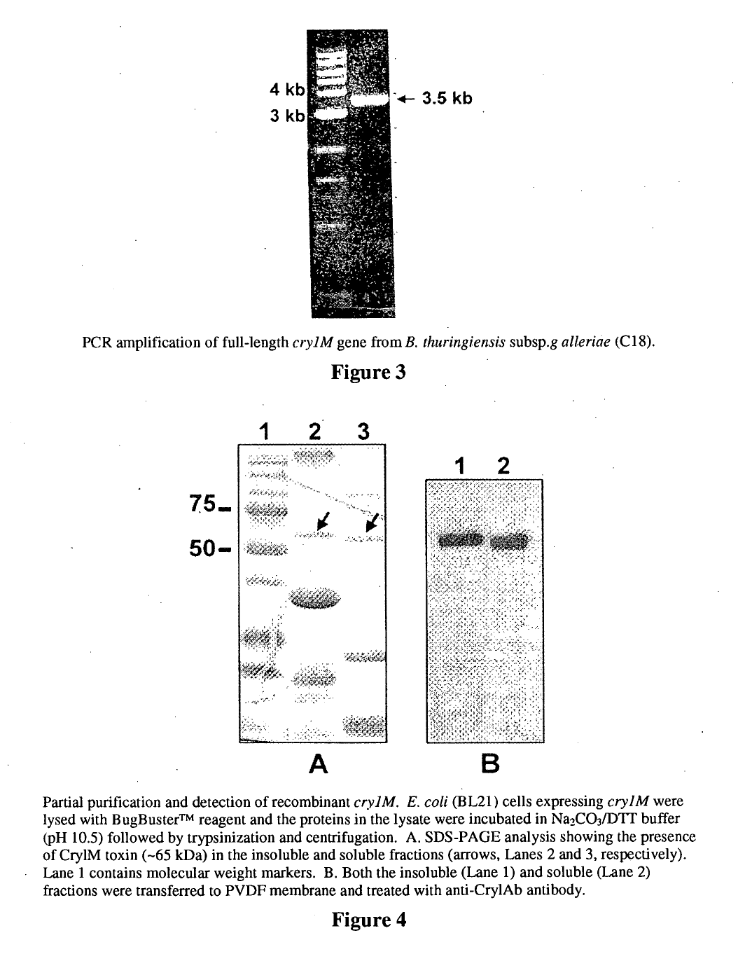

A 3.5 kb fragment was obtained as shown on the gel set forth in FIG. 3. This nucleic acid was sequenced with the results along with deduced amino acid sequence shown in FIG. 1. The amino acid sequence is

MEISDQNQYIPYNCLNNPESEIFNARNSNFGLVSQVSSGLTRFLLEAAVP
EAGFALGLFDIIWGALGVDQWSLFLRQIEQLIRQEITELERNRATAILTG
LSSSYNLYVEALREWENDPNNPASQERVRTRFRLTDDAIVTGLPTLAIRN
LEVVNLSVYTQAANLHLSLLRDAVYFGERWGLTQANIEDLYTRLTSNIQE
YSDHCARWYNQGLNEIGGISRRYLDFQRDFTISVLDIVALFPNYDIRTYP
IPTQSQFTREIYTSPVVAGNINFGLSIANVLRAPHLMDFIDRIVIYTNSV
RSTPYWAGHEVILRRTGQGQGNEIRFPLYGVAANAEPPVTIRPTGFTDEQ
RQWYRARSRVVLFRSSGQDFSLVDAVGFLTIFSAVSIYRNGFGFNTDTID
EIPIEGTDPFTGYSHRLCHVGFLASSPFISQYARAPIFSWTHRSATLTNT
MAPDVITQIPLVKAFNLHSGATIVKGPGFTGGDILRRTNVGSFGDMRVNI
TAPLSQRYRVRIRYASTTDLQFYTNINGTTINIGNFSSTMDSGDDLQYGR
FRVAGFTTPFTFSDANSTFTIGAFGFSPNNEVYIDRIEFVPAEVTFEAEY
DLEKAQKAVNALFTSSNQ.

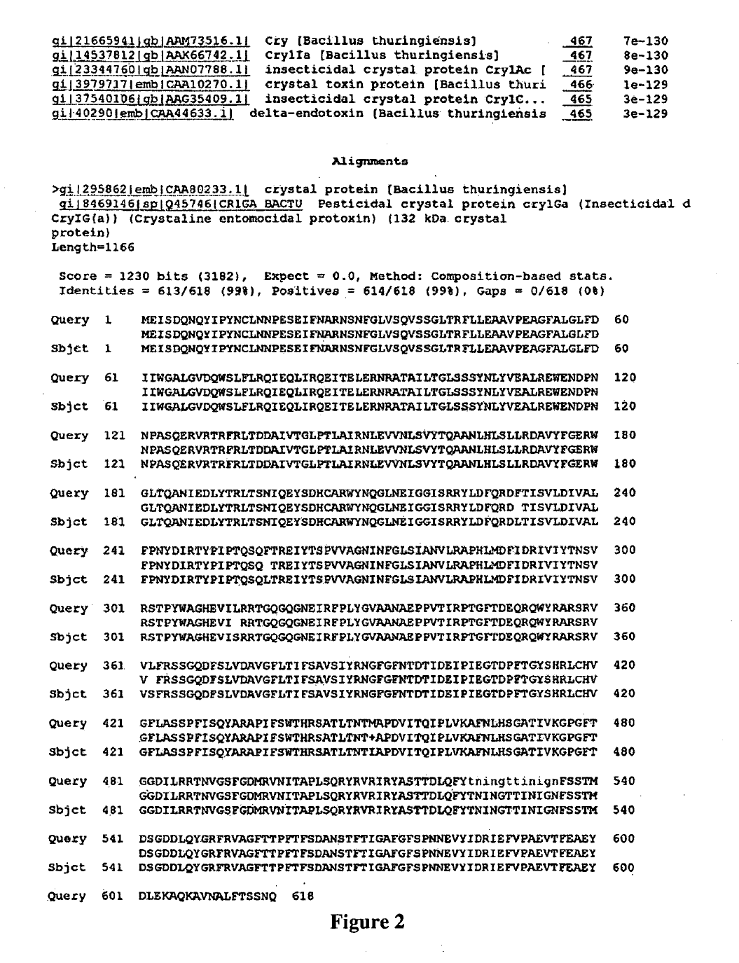

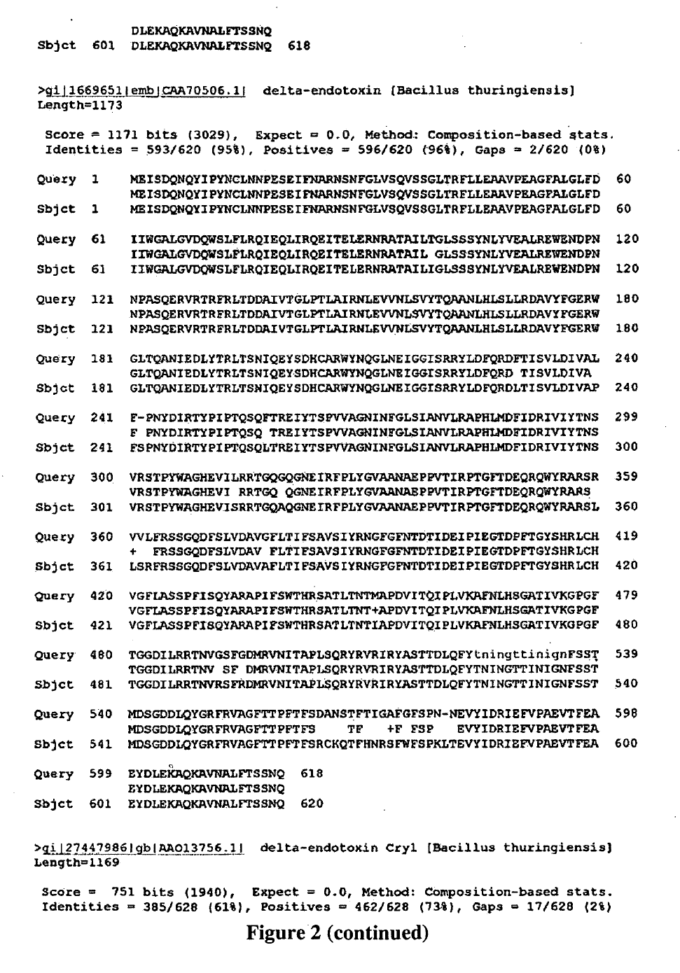

This sequence was compared with proteins retrieved in a BLAST search as shown in FIG. 2.

When the deduced amino acid sequence of Cry1M was BLASTed against available sequences in the database, two proteins (accession# CAA80233 and accession# CAA70506) showed the most similarity with Cry1M. Cry1M and CAA80233 share 99% amino acid identity with a difference of four amino acids, F₂₃₀-L, F₂₅₇-L, L₃₁₃-S and L₃₆₂-S. Cry1M and CAA70506 were 95% identical. There is a difference of twenty-two amino acid between the two proteins—ten of these amino acids are in a region of the protein responsible for insect host specificity.

### EXAMPLE 2

### Recombinant Production of Cry1M Protein

The coding sequence for Cry1M was inserted into the expression vector pQE-30UA (QIAGEN), which vector was used to transfect E. coli BL21 cells. Two hundred (200) ml of LB broth containing ampicillin (100 μg /ml) was inoculated with 2 ml of an overnight culture of BL21(DE3) that harbors the cloned cry1M gene in pQU-30UA plasmid vector. Cells were allowed to grow at 37° C. to mid-exponential phase growth (approximately 4 hours). The culture was then induced with 1 mM IPTG and cells continued to grow for an additional 12 hours.

A cell pellet was collected by centrifugation at 9,000 rpm for 15 min and resuspended in BugBuster™ reagent (Novagen). Lysozyme (250 μg/ml) and Benzonase® (Novagen) (25 units/ml) were added to the cell lysate. Inclusion bodies were separated from the soluble protein fraction by centrifugation and washed several times with diluted (1:10) BugBuster™ as recommended by the manufacturer (Novagen). The inclusion bodies were re-suspended in NaHCO₃ buffer (100 mM; pH 8.0) that contained 0.2% βME. Cry1M protoxin was activated with trypsin (1 mg/ml) at 30° C. for 90 min. All protein fractions were analyzed by SDS-PAGE.

The Cry1M toxin, with a molecular weight of about 65 kDa was detected in both the insoluble and soluble fractions. In FIG. 4a, Lane 1 represents molecular weight markers, Lane 2 represents the treated insoluble fraction and Lane 3 represents the treated soluble fraction.

FIG. 4b shows the results when both the insoluble and soluble fractions were transferred to PVDF membrane and treated with anti-Cry1Ab antibody. Lane 1 shows the insoluble fraction and Lane 2 shows the soluble fraction.

### EXAMPLE 3

### Toxicity of Recombinant Cry1M

Crude extracts of recombinant E. coli cells harboring Cry1M-pQU30A were applied uniformly to the surface of artificial diet on which first-instar larvae of tobacco homworm (Manduca sexta) were placed. Mortality was recorded after 72 hours.

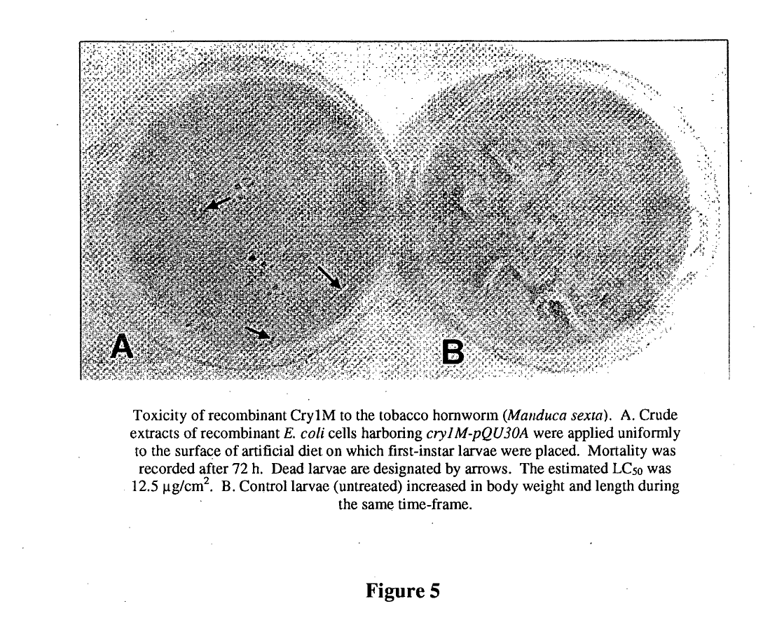

In FIG. 5a, dead larvae are designated by arrows, and FIG. 5b shows that control larvae where the artificial diet surface did not contain the extract increased in body weight and length during the 72-hour time frame.

The estimated LC₅₀ was 12.5 μg/cm².

1. A purified and isolated protein having the 618 amino acid sequence set forth in FIG. 1 or variant thereof that is at least 90% identical thereto and which retains insecticidal and/or nematicidal activity.

2. The protein of claim 1 that has the 618 amino acid sequence set forth in FIG. 1.

3. A nucleic acid molecule that comprises a nucleotide sequence encoding the protein of claim 1 or the complement of said nucleotide sequence.

4. The nucleic acid molecule of claim 3 which further comprises control sequences for expression operably linked to said nucleotide sequence.

5. The nucleic acid molecule of claim 3, wherein said control sequences are operable to effect expression in unicellular organisms.

6. The nucleic acid molecule of claim 3, wherein said control sequences are operable to effect expression in plants.

7. Recombinant host cells that have been modified to contain the nucleic acid molecule of claim 5.

8. The cells of claim 7 that are plant cells.

9. An intact plant that has been modified to contain the expression system of claim 6.

10. A method to produce an insecticidal and/or nematicidal protein which method comprises culturing the cells of claim 7.

11. A method to produce an insecticidal protein which method comprises culturing the plant of claim 8.

12. Antibodies or fragments thereof that are specifically immunoreactive with the protein of claim 1.

13. An insecticidal and/or nematicidal composition which comprises the protein of claim 1 in admixture with excipients suitable for such compositions.

14. A method to determine the susceptibility of an insect or nematode to an insecticidal toxin which method comprises contacting said insect or nematode with the protein of claim 1 and assessing the effect of said protein on the insect or nematode.
